(19) United States
(12) Patent Application Publication
(10) Pub. No.: US 2023/0063142 A1
(43) Pub. Date: Mar. 2, 2023

(54) Replacement Heart Valve Implant and Expandable Framework for Replacement Heart Valve Implant

(71) Applicant: BOSTON SCIENTIFIC SCIMED, INC., Maple Grove, MN (US)

(72) Inventors: Tim O'Connor, Galway (IE); Joseph Murphy, Galway (IE); Declan Loughnane, Galway (IE); Dongming Hou, Plymouth, MN (US); Shane Conway, Longford (IE)

(73) Assignee: BOSTON SCIENTIFIC SCIMED, INC., MAPLE GROVE, MN (US)

(21) Appl. No.: 17/894,379

(22) Filed: Aug. 24, 2022

Related U.S. Application Data
(60) Provisional application No. 63/236,977, filed on Aug. 25, 2021.

(51) Int. Cl. A61F 2/24

(52) U.S. Cl. CPC A61F 2/2418; A61F 2220/0075; A61F 2250/0036

(57) ABSTRACT

A replacement heart valve implant may include an expandable framework having a plurality of frame struts defining a lattice structure, each frame strut having a thickness in a radial direction from a central longitudinal axis; and a plurality of valve leaflets coupled to the expandable framework. The plurality of frame struts defines a lower crown proximate an inflow end of the lattice structure and upper crown proximate an outflow end of the lattice structure and a plurality of stabilization arches extending downstream from the outflow end of the lattice structure. The lattice structure includes a first circumferential row of x-connectors upstream of the upper crown and a second circumferential row of x-connectors downstream of the lower crown. The thickness of at least some frame struts connecting the first and second circumferential rows of x-connectors is less than the thickness of other frame struts of the plurality of frame struts.

### CROSS-REFERENCE TO RELATED APPLICATIONS

This application claims the benefit of priority of U.S. Provisional Application No. 63/236,977 filed Aug. 25, 2021, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to a replacement heart valve implant and/or an expandable framework for use in a replacement heart valve implant.

### BACKGROUND

A wide variety of intracorporeal medical devices have been developed for medical use, for example, intravascular use. Some of these devices include guidewires, catheters, medical device systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In one example, an expandable framework for use in a replacement heart valve implant, may comprise a plurality of frame struts defining a lattice structure around a central longitudinal axis, each frame strut having a thickness in a radial direction from the central longitudinal axis. The plurality of frame struts defines a lower crown proximate an inflow end of the lattice structure and upper crown proximate an outflow end of the lattice structure and a plurality of stabilization arches extending downstream from the outflow end of the lattice structure. The lattice structure includes a first circumferential row of x-connectors upstream of the upper crown and a second circumferential row of x-connectors downstream of the lower crown. The thickness of at least some frame struts connecting the first circumferential row of x-connectors to the second circumferential row of x-connectors is less than the thickness of other frame struts of the plurality of frame struts.

In addition or alternatively to any example described herein, the thickness of the at least some frame struts connecting the first circumferential row of x-connectors to the second circumferential row of x-connectors is less than the thickness of frame struts downstream of the first circumferential row of x-connectors.

In addition or alternatively to any example described herein, the thickness of the at least some frame struts connecting the first circumferential row of x-connectors to the second circumferential row of x-connectors is less than the thickness of all frame struts downstream of the first circumferential row of x-connectors.

In addition or alternatively to any example described herein, the thickness of the at least some frame struts connecting the first circumferential row of x-connectors to the second circumferential row of x-connectors is less than the thickness of frame struts upstream of the second circumferential row of x-connectors.

In addition or alternatively to any example described herein, the thickness of the at least some frame struts connecting the first circumferential row of x-connectors to the second circumferential row of x-connectors is less than the thickness of all frame struts upstream of the second circumferential row of x-connectors.

In addition or alternatively to any example described herein, the thickness of the at least some frame struts connecting the first circumferential row of x-connectors to the second circumferential row of x-connectors tapers radially inward toward a medial portion of the at least some frame struts connecting the first circumferential row of x-connectors to the second circumferential row of x-connectors.

In addition or alternatively to any example described herein, the thickness of the at least some frame struts connecting the first circumferential row of x-connectors to the second circumferential row of x-connectors tapers from a first thickness at a first position adjacent the first circumferential row of x-connectors and a second thickness at a second position adjacent the second circumferential row of x-connectors to a minimum thickness at a third position disposed longitudinally between the first position and the second position. The minimum thickness is less than the first thickness and the second thickness.

In addition or alternatively to any example described herein, the at least some frame struts connecting the first circumferential row of x-connectors to the second circumferential row of x-connectors includes all frame struts of the plurality of frame struts directly connecting the first circumferential row of x-connectors to the second circumferential row of x-connectors.

In addition or alternatively to any example described herein, the upper crown defines a first maximum outer extent of the lattice structure, and the lower crown defines a second maximum outer extent of the lattice structure. The at least some frame struts connecting the first circumferential row of x-connectors to the second circumferential row of x-connectors defines a third maximum outer extent of the lattice structure less than the first maximum outer extent and the second maximum outer extent.

In addition or alternatively to any example described herein, a replacement heart valve implant may comprise an expandable framework comprising a plurality of frame struts defining a lattice structure around a central longitudinal axis, each frame strut having a thickness in a radial direction from the central longitudinal axis; and a plurality of valve leaflets coupled to the expandable framework. The plurality of frame struts defines a lower crown proximate an inflow end of the lattice structure and upper crown proximate an outflow end of the lattice structure and a plurality of stabilization arches extending downstream from the outflow end of the lattice structure. The lattice structure includes a first circumferential row of x-connectors upstream of the upper crown and a second circumferential row of x-connectors downstream of the lower crown. The thickness of at least some frame struts connecting the first circumferential row of x-connectors to the second circumferential row of x-connectors is less than the thickness of other frame struts of the plurality of frame struts.

In addition or alternatively to any example described herein, the plurality of valve leaflets is configured to substantially restrict fluid from flowing through the replacement heart valve implant in a closed position.

In addition or alternatively to any example described herein, the plurality of valve leaflets is fixedly attached to the expandable framework at a plurality of commissures disposed adjacent the plurality of stabilization arches.

In addition or alternatively to any example described herein, the plurality of commissures is disposed longitudinally between the plurality of stabilization arches and the upper crown.

In addition or alternatively to any example described herein, the replacement heart valve implant may further comprise an outer skirt disposed on an abluminal surface of the expandable framework.

In addition or alternatively to any example described herein, an expandable framework for use in a replacement heart valve implant may comprise a plurality of frame struts defining a lattice structure around a central longitudinal axis, each frame strut having a thickness in a radial direction from the central longitudinal axis. The plurality of frame struts defines a lower crown proximate an inflow end of the lattice structure and upper crown proximate an outflow end of the lattice structure and a plurality of stabilization arches extending downstream from the outflow end of the lattice structure. The lattice structure includes a first circumferential row of x-connectors and a second circumferential row of x-connectors longitudinally spaced apart from the first circumferential row of x-connectors. The thickness of at least some frame struts directly connecting the first circumferential row of x-connectors to the second circumferential row of x-connectors varies.

In addition or alternatively to any example described herein, the thickness of at least some frame struts directly connecting the first circumferential row of x-connectors to the second circumferential row of x-connectors varies in a longitudinal direction.

In addition or alternatively to any example described herein, the thickness of each frame strut directly connecting the first circumferential row of x-connectors to the second circumferential row of x-connectors varies.

In addition or alternatively to any example described herein, the thickness of each frame strut directly connecting the first circumferential row of x-connectors to the second circumferential row of x-connectors varies in a longitudinal direction.

In addition or alternatively to any example described herein, the thickness of the at least some frame struts directly connecting the first circumferential row of x-connectors to the second circumferential row of x-connectors tapers radially inward toward a medial portion of the at least some frame struts directly connecting the first circumferential row of x-connectors to the second circumferential row of x-connectors.

In addition or alternatively to any example described herein, the thickness of the at least some frame struts directly connecting the first circumferential row of x-connectors to the second circumferential row of x-connectors tapers radially inward from the first circumferential row of x-connectors toward the second circumferential row of x-connectors and the thickness of the at least some frame struts directly connecting the first circumferential row of x-connectors to the second circumferential row of x-connectors tapers radially inward from the second circumferential row of x-connectors toward the first circumferential row of x-connectors.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a native heart valve;

FIG. 2 illustrates selected aspects of a replacement heart valve implant;

FIGS. 3-4 illustrate selected aspects of an expandable framework for use in a replacement heart valve implant;

FIG. 5 is a partial cross-sectional view illustrating selected aspects of the expandable framework positioned within the native heart valve;

FIGS. 6-8 illustrate selected aspects of a method of manufacturing the expandable framework; and

FIG. 9 illustrates selected aspects of an alternative configuration of the expandable framework.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate example embodiments of the disclosure but not limit the disclosure. Those skilled in the art will recognize that the various elements described and/or shown may be arranged in various combinations and configurations without departing from the scope of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. For example, failure of the aortic valve or the mitral valve can have a serious effect on a human and could lead to a serious health condition and/or death if not dealt with properly. Treatment of defective heart valves poses other challenges in that the treatment often requires the repair or outright replacement of the defective heart valve. Such therapies may be highly invasive to the patient. Disclosed herein is an apparatus, system, and/or method that may be used in a portion of the cardiovascular system in order to diagnose, treat, and/or repair the system. In some embodiments, the apparatus, system, and/or method disclosed herein may be used before and/or during a procedure to diagnose, treat, and/or repair a defective heart valve (e.g., the aortic valve, the mitral valve, etc.). In addition, a replacement heart valve implant may be delivered percutaneously and thus may be much less invasive to the patient. The apparatus, system, and/or method disclosed herein may also provide other desirable features and/or benefits as described below.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the leaflet”, “the frame strut”, or other features may be equally referred to all instances and quantities beyond one of said feature unless clearly stated to the contrary. As such, it will be understood that the following discussion may apply equally to any and/or all of the components for which there are more than one within the replacement heart valve implant and/or the apparatus unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The systems, devices, and/or methods disclosed herein may provide a number of desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices disclosed herein may have applications and uses in other portions of a patient's anatomy, such as but not limited to, arteries, veins, and/or other body lumens.

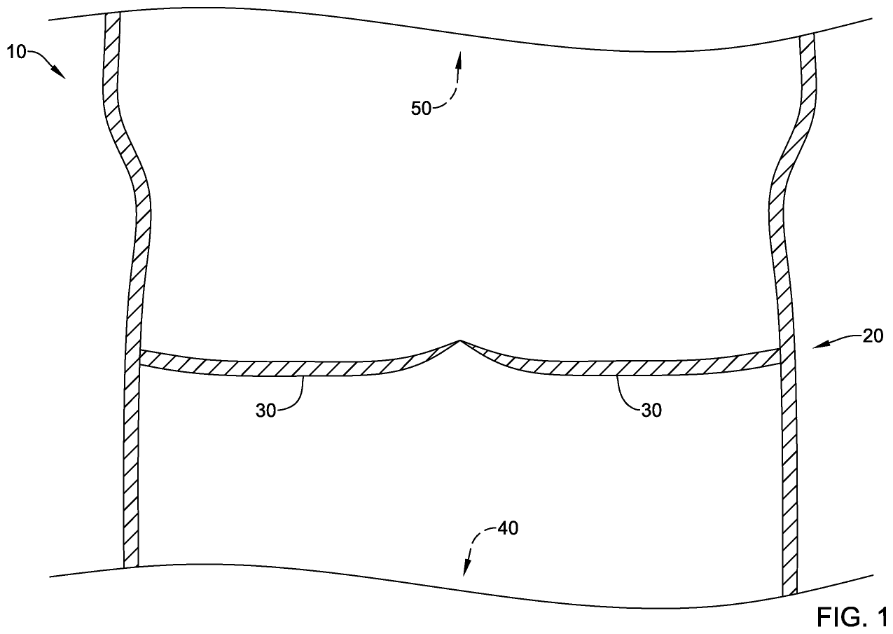

FIG. 1 illustrates a native heart valve 10 (e.g., a native aortic valve, etc.). The native heart valve 10 may include an annulus 20 defined at least partially by one or more walls of the native heart, and a plurality of leaflets 30 extending radially inward from the annulus 20. During systole, the plurality of leaflets 30 may shift to an open position to permit blood to flow through the native heart valve 10 (e.g., from the left ventricle 40, through the native heart valve 10, and downstream into the aortic arch 50). During diastole, the plurality of leaflets 30 may shift to a closed position to prevent blood from flowing through the native heart valve 10. In some cases, a diseased and/or defective native heart valve 10 may have and/or include calcification in, on, and/or around the annulus 20.

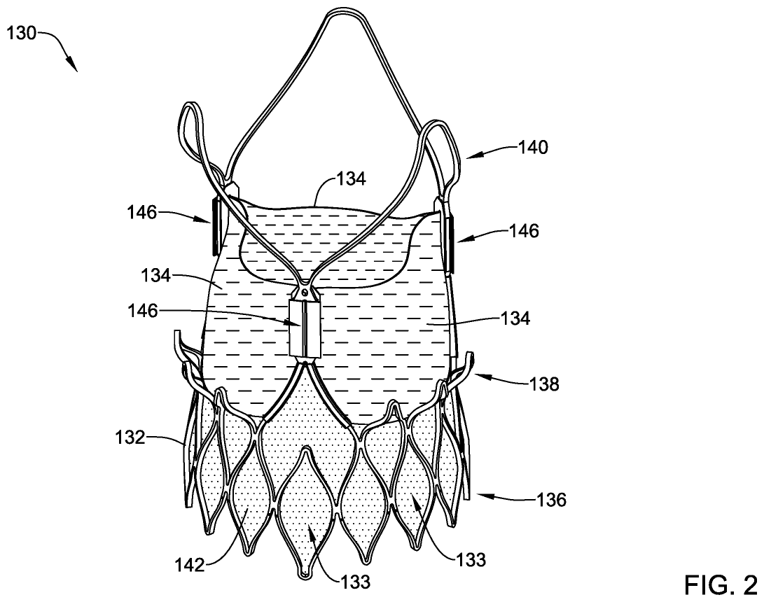

FIG. 2 illustrates selected aspects of a replacement heart valve implant 130. It should be appreciated that the replacement heart valve implant 130 can be any type of heart valve (e.g., a mitral valve, an aortic valve, etc.). In use, the replacement heart valve implant 130 may be implanted (e.g., surgically or through transcatheter delivery) in a mammalian heart. The replacement heart valve implant 130 can be configured to allow one-way flow through the replacement heart valve implant 130 from an inflow end to an outflow end.

The replacement heart valve implant 130 may include an expandable framework 132 defining a central lumen which, in some embodiments, may be substantially cylindrical. The side of the expandable framework 132 and other components facing the central lumen can be referred to as the luminal surface or luminal side. The opposite or outer side of the expandable framework 132 and other components (e.g., facing away from the central lumen) can be referred to as the abluminal surface or abluminal side. In some embodiments, the expandable framework 132 may have a substantially circular cross-section. In some embodiments, the expandable framework 132 can have a non-circular (e.g., D-shaped, elliptical, etc.) cross-section. In some embodiments, a non-circular expandable framework can be used to repair a mitral valve or another non-circular valve in the patient's heart or body. Some suitable but non-limiting examples of materials that may be used to form the expandable framework 132, including but not limited to metals and metal alloys, composites, ceramics, polymers, and the like, are described below.

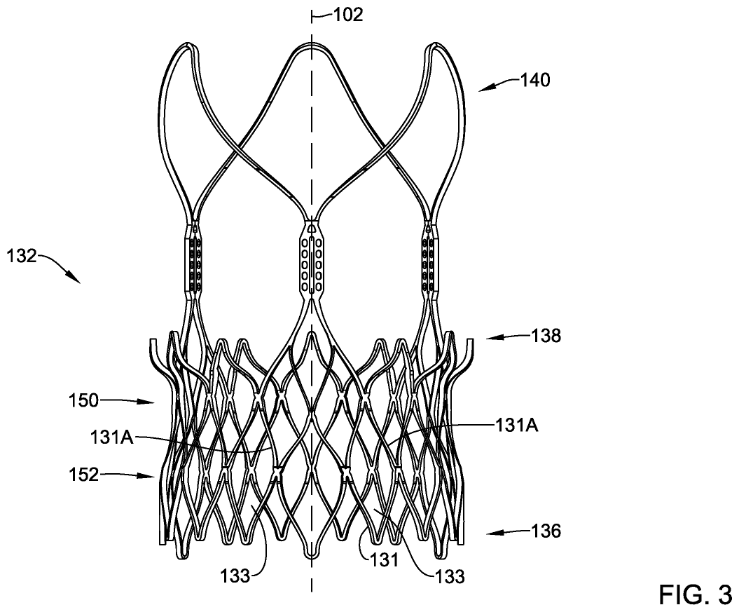

The expandable framework 132 may be configured to shift from a collapsed configuration to an expanded configuration. In some embodiments, the expandable framework 132 may be self-expanding. In some embodiments, the expandable framework 132 may be self-biased toward the expanded configuration. In some embodiments, the expandable framework 132 may be mechanically expandable. In some embodiments, the expandable framework 132 may be balloon expandable. Other configurations are also contemplated. As seen in FIG. 3, the expandable framework 132 may include a plurality of frame struts 131. In some embodiments, the plurality of frame struts 131 may define a lattice structure disposed and/or extending around a central longitudinal axis 102. In some embodiments, the plurality of frame struts 131 may define a plurality of interstices 133 (e.g., openings) between adjacent frame struts and/or through the expandable framework 132 from the luminal side to the abluminal side.

In some embodiments, the expandable framework 132 and/or the plurality of frame struts 131 may define a lower crown 136 proximate an inflow end of the lattice structure, an upper crown 138 proximate an outflow end of the lattice structure, and a plurality of stabilization arches 140 extending downstream from the outflow end of the lattice structure. In some embodiments, the lower crown 136 may be disposed at the inflow end of the lattice structure. In some embodiments, the upper crown 138 may be disposed at the outflow end of the lattice structure. In some embodiments, the plurality of stabilization arches 140 may extend downstream of and/or away from the upper crown 138 in a direction opposite the lower crown 136. In some embodiments, the upper crown 138 may be disposed longitudinally and/or axially between the lower crown 136 and the plurality of stabilization arches 140.

In some embodiments, the expandable framework 132 and/or the lattice structure may include a first circumferential row of x-connectors 150 upstream of the upper crown 138 and a second circumferential row of x-connectors 152 downstream of the lower crown 136. The first circumferential row of x-connectors 150 and/or the second circumferential row of x-connectors 152 may connect adjacent frame struts of the plurality of frame struts 131. In some embodiments, the first circumferential row of x-connectors 150 and/or the second circumferential row of x-connectors 152 may serve as a junction or joint between adjacent frame struts of the plurality of frame struts 131. In some embodiments, the first circumferential row of x-connectors 150 and/or the second circumferential row of x-connectors 152 may permit some degree of movement between adjacent struts of the plurality of frame struts 131 as the expandable framework 132 and/or the lattice structure shifts from the collapsed configuration to the expanded configuration. For example, an angle between adjacent frame struts of the plurality of frame struts 131 may change from a first angle in the collapsed configuration to a second angle in the expanded configuration. Other configurations are also contemplated.

In some embodiments, the first circumferential row of x-connectors 150 and/or the second circumferential row of x-connectors 152 may extend continuously and/or completely around the expandable framework 132 and/or the lattice structure. In some embodiments, the first circumferential row of x-connectors 150 and/or the second circumferential row of x-connectors 152 may be discontinuous and/or may extend partially around the expandable framework 132 and/or the lattice structure. Other configurations are also contemplated.

In some embodiments, the first circumferential row of x-connectors 150 and the second circumferential row of x-connectors 152 may be disposed longitudinally between the upper crown 138 and the lower crown 136. In some embodiments, the first circumferential row of x-connectors 150 may be disposed downstream of the second circumferential row of x-connectors 152. In some embodiments, the second circumferential row of x-connectors 152 may be disposed upstream of the first circumferential row of x-connectors 150.

In some embodiments, one or more additional circumferential rows of x-connectors may be provided and/or disposed longitudinally between the upper crown 138 and the lower crown 136. In at least some embodiments, the first circumferential row of x-connectors 150 may be disposed adjacent the second circumferential row of x-connectors 152, such that no other circumferential rows of x-connectors are disposed longitudinally between the first circumferential row of x-connectors 150 and the second circumferential row of x-connectors 152.

In some embodiments, the second circumferential row of x-connectors 152 may be longitudinally spaced apart from the first circumferential row of x-connectors 150 by at least some frame struts 131A of the plurality of frame struts 131. In some embodiments, at least some frame struts 131A of the plurality of frame struts 131 connect the first circumferential row of x-connectors 150 to the second circumferential row of x-connectors 152. In some embodiments, at least some frame struts 131A of the plurality of frame struts 131 directly connect the first circumferential row of x-connectors 150 to the second circumferential row of x-connectors 152. As such, the at least some frame struts 131A of the plurality of frame struts 131 connecting the first circumferential row of x-connectors 150 to the second circumferential row of x-connectors 152 may be directly connected to the first circumferential row of x-connectors 150 and the second circumferential row of x-connectors 152. In some embodiments, the at least some frame struts 131A of the plurality of frame struts 131 connecting the first circumferential row of x-connectors 150 to the second circumferential row of x-connectors 152 may include all frame struts directly connecting the first circumferential row of x-connectors 150 to the second circumferential row of x-connectors 152.

Each frame strut of the plurality of frame struts 131 and each x-connector of the first circumferential row of x-connectors 150 and the second circumferential row of x-connectors 152 may have a thickness in a radial direction from the central longitudinal axis 102. Similarly, each frame struts of the plurality of frame struts 131 may have a width defined in a circumferential direction around the central longitudinal axis 102. In at least some embodiments, the thickness of the plurality of frame struts 131 may be greater than the width of the plurality of frame struts 131.

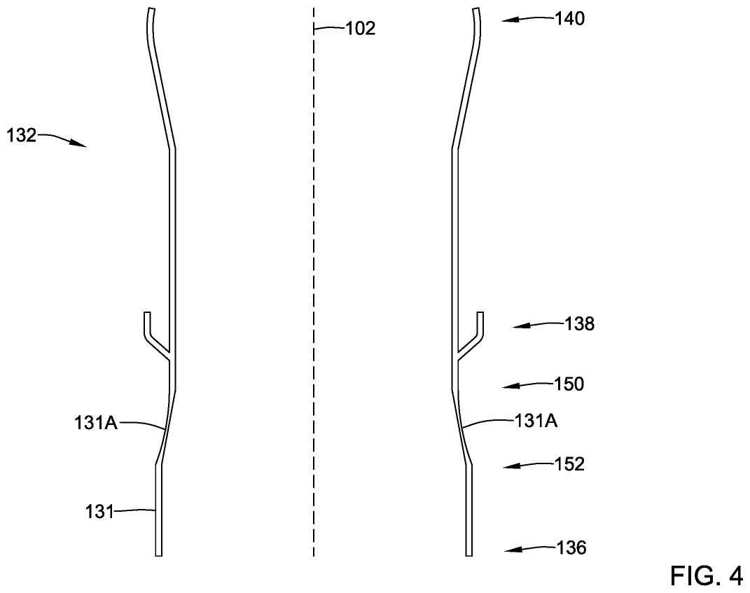

As shown in the partial cross-section of FIG. 4, in some embodiments, the thickness of at least some frame struts 131A of the plurality of frame struts 131 connecting the first circumferential row of x-connectors 150 to the second circumferential row of x-connectors 152 may be less than the thickness of other frame struts of the plurality of frame struts 131. In some embodiments, the thickness of the at least some frame struts 131A of the plurality of frame struts 131 connecting the first circumferential row of x-connectors 150 to the second circumferential row of x-connectors 152 may be less than the thickness of frame struts downstream of the first circumferential row of x-connectors 150. In some embodiments, the thickness of the at least some frame struts 131A of the plurality of frame struts 131 connecting the first circumferential row of x-connectors 150 to the second circumferential row of x-connectors 152 may be less than the thickness of all frame struts downstream of the first circumferential row of x-connectors 150. In some embodiments, the thickness of the at least some frame struts 131A of the plurality of frame struts 131 connecting the first circumferential row of x-connectors 150 to the second circumferential row of x-connectors 152 may be less than the thickness of frame struts upstream of the second circumferential row of x-connectors 152. In some embodiments, the thickness of the at least some frame struts 131A of the plurality of frame struts 131 connecting the first circumferential row of x-connectors 150 to the second circumferential row of x-connectors 152 may be less than the thickness of all frame struts upstream of the second circumferential row of x-connectors 152.

In some embodiments, the thickness of at least some frame struts 131A of the plurality of frame struts 131 connecting the first circumferential row of x-connectors 150 to the second circumferential row of x-connectors 152 varies. In some embodiments, the thickness of at least some frame struts 131A of the plurality of frame struts 131 connecting the first circumferential row of x-connectors 150 to the second circumferential row of x-connectors 152 varies in a longitudinal direction. In some embodiments, the thickness of each frame strut of the plurality of frame struts 131 connecting the first circumferential row of x-connectors 150 to the second circumferential row of x-connectors 152 varies. In some embodiments, the thickness of each frame strut of the plurality of frame struts 131 connecting the first circumferential row of x-connectors 150 to the second circumferential row of x-connectors 152 varies in the longitudinal direction.

In some embodiments, the thickness of at least some frame struts 131A of the plurality of frame struts 131 directly connecting the first circumferential row of x-connectors 150 to the second circumferential row of x-connectors 152 varies. In some embodiments, the thickness of at least some frame struts 131A of the plurality of frame struts 131 directly connecting the first circumferential row of x-connectors 150 to the second circumferential row of x-connectors 152 varies in the longitudinal direction. In some embodiments, the thickness of each frame strut of the plurality of frame struts 131 directly connecting the first circumferential row of x-connectors 150 to the second circumferential row of x-connectors 152 varies. In some embodiments, the thickness of each frame strut of the plurality of frame struts 131 directly connecting the first circumferential row of x-connectors 150 to the second circumferential row of x-connectors 152 varies in the longitudinal direction.

In some embodiments, the thickness of at least some frame struts 131A of the plurality of frame struts 131 connecting the first circumferential row of x-connectors 150 to the second circumferential row of x-connectors 152 tapers radially inward toward a medial portion of the at least some frame struts 131A of the plurality of frame struts 131 connecting the first circumferential row of x-connectors 150 to the second circumferential row of x-connectors 152. In some embodiments, the thickness of each frame strut of the plurality of frame struts 131 connecting the first circumferential row of x-connectors 150 to the second circumferential row of x-connectors 152 tapers radially inward toward a medial portion of each frame strut of the plurality of frame struts 131 connecting the first circumferential row of x-connectors 150 to the second circumferential row of x-connectors 152. In some embodiments, the thickness of the at least some frame struts 131A of the plurality of frame struts 131 connecting the first circumferential row of x-connectors 150 to the second circumferential row of x-connectors 152 tapers radially inward from the first circumferential row of x-connectors 150 toward the second circumferential row of x-connectors 152 and the thickness of the at least some frame struts 131A of the plurality of frame struts 131 connecting the first circumferential row of x-connectors 150 to the second circumferential row of x-connectors 152 tapers radially inward from the second circumferential row of x-connectors 152 toward the first circumferential row of x-connectors 150.

In some embodiments, the thickness of at least some frame struts 131A of the plurality of frame struts 131 directly connecting the first circumferential row of x-connectors 150 to the second circumferential row of x-connectors 152 tapers radially inward toward a medial portion of the at least some frame struts 131A of the plurality of frame struts 131 directly connecting the first circumferential row of x-connectors 150 to the second circumferential row of x-connectors 152. In some embodiments, the thickness of each frame strut of the plurality of frame struts 131 directly connecting the first circumferential row of x-connectors 150 to the second circumferential row of x-connectors 152 tapers radially inward toward a medial portion of each frame strut of the plurality of frame struts 131 directly connecting the first circumferential row of x-connectors 150 to the second circumferential row of x-connectors 152. In some embodiments, the thickness of the at least some frame struts 131A of the plurality of frame struts 131 directly connecting the first circumferential row of x-connectors 150 to the second circumferential row of x-connectors 152 tapers radially inward from the first circumferential row of x-connectors 150 toward the second circumferential row of x-connectors 152 and the thickness of the at least some frame struts 131A of the plurality of frame struts 131 directly connecting the first circumferential row of x-connectors 150 to the second circumferential row of x-connectors 152 tapers radially inward from the second circumferential row of x-connectors 152 toward the first circumferential row of x-connectors 150.

In some embodiments, the thickness of at least some frame struts 131A of the plurality of frame struts 131 connecting the first circumferential row of x-connectors 150 to the second circumferential row of x-connectors 152 tapers from a first thickness at a first position adjacent the first circumferential row of x-connectors 150 and a second thickness at a second position adjacent the second circumferential row of x-connectors 152 to a minimum thickness at a third position disposed longitudinally between the first position and the second position. In some embodiments, the thickness of each frame strut of the plurality of frame struts 131 connecting the first circumferential row of x-connectors 150 to the second circumferential row of x-connectors 152 tapers from a first thickness at a first position adjacent the first circumferential row of x-connectors 150 and a second thickness at a second position adjacent the second circumferential row of x-connectors 152 to a minimum thickness at a third position disposed longitudinally between the first position and the second position. The minimum thickness at the third position may be less than the first thickness and the second thickness.

In some embodiments, the thickness of at least some frame struts 131A of the plurality of frame struts 131 directly connecting the first circumferential row of x-connectors 150 to the second circumferential row of x-connectors 152 tapers from a first thickness at a first position adjacent the first circumferential row of x-connectors 150 and a second thickness at a second position adjacent the second circumferential row of x-connectors 152 to a minimum thickness at a third position disposed longitudinally between the first position and the second position. In some embodiments, the thickness of each frame strut of the plurality of frame struts 131 directly connecting the first circumferential row of x-connectors 150 to the second circumferential row of x-connectors 152 tapers from a first thickness at a first position adjacent the first circumferential row of x-connectors 150 and a second thickness at a second position adjacent the second circumferential row of x-connectors 152 to a minimum thickness at a third position disposed longitudinally between the first position and the second position. The minimum thickness at the third position may be less than the first thickness and the second thickness.

In some embodiments, the upper crown 138 may define a first maximum outer extent of the lattice structure, and the lower crown 136 may define a second maximum outer extent of the lattice structure. In some embodiments, the at least some frame struts 131A of the plurality of frame struts 131 connecting the first circumferential row of x-connectors 150 to the second circumferential row of x-connectors 152 may define a third maximum outer extent of the lattice structure less than the first maximum outer extent. In some embodiments, the third maximum outer extent of the lattice structure may be less than the second maximum outer extent. In some embodiments, the third maximum outer extent of the lattice structure may be less than the first maximum outer extent and the second maximum outer extent.

Returning now to FIG. 2, the replacement heart valve implant 130 may include a plurality of valve leaflets 134 disposed within the central lumen. The plurality of valve leaflets 134 may be coupled, secured, and/or fixedly attached to the expandable framework 132. Each of the plurality of valve leaflets 134 may include a root edge coupled to the expandable framework 132 and a free edge (e.g., a coaptation edge) movable relative to the root edge to coapt with the coaptation edges of the other leaflets along a coaptation region. In some embodiments, the plurality of valve leaflets 134 can be integrally formed with each other, such that the plurality of valve leaflets 134 is formed as a single unitary and/or monolithic unit. In some embodiments, a “root edge” can be a formed edge, such as when the plurality of valve leaflets 134 is formed in place on the expandable framework 132. In some embodiments, the plurality of valve leaflets 134 may be formed integrally with other structures such as an inner skirt 142 and/or an outer skirt (not shown), base structures, liners, or the like and in those circumstances the “root edge” is not a cut or otherwise divided edge, but rather is the location opposite the free edge where each of the plurality of valve leaflets 134 meets those other structures.

The plurality of valve leaflets 134 may be configured to substantially restrict fluid from flowing through the replacement heart valve implant 130 in a closed position. For example, in some embodiments, the free edges of the plurality of valve leaflets 134 may move into coaptation with one another in the closed position to substantially restrict fluid from flowing through the replacement heart valve implant 130. Specifically, the plurality of valve leaflets 134 may coapt to fill up or close the central lumen of the replacement heart valve implant 130 thereby impeding the flow of fluid through the replacement heart valve implant 130. The free edges of the plurality of valve leaflets 134 may be move apart from each other in an open position to permit fluid flow through the replacement heart valve implant 130. Specifically, the plurality of valve leaflets 134 may move apart from each other to open the central lumen of the replacement heart valve implant 130 thereby permitting the flow of fluid through the replacement heart valve implant 130. In FIG. 2, the plurality of valve leaflets 134 is shown in the open position or in a partially open position (e.g., a neutral position) that the plurality of valve leaflets 134 may move to when unbiased by fluid flow.

Each of the plurality of valve leaflets 134 may further include two connection portions. One connection portion can be disposed on either end of the free edge of its respective leaflet such that the connection portions are contacting or adjacent to the expandable framework 132 at a plurality of commissures 146 disposed adjacent the plurality of stabilization arches 140. In some embodiments, the plurality of valve leaflets 134 may be secured and/or fixedly attached to the expandable framework 132 at the plurality of commissures 146 disposed adjacent the plurality of stabilization arches 140. The free edges of the plurality of valve leaflets 134 may extend between the plurality of commissures 146.

In some embodiments, the plurality of commissures 146 may be disposed at a base of the plurality of stabilization arches 140. In some embodiments, each of the plurality of commissures 146 may join circumferentially adjacent stabilization arches of the plurality of stabilization arches 140 together. In some embodiments, the plurality of commissures 146 may be disposed longitudinally between the plurality of stabilization arches 140 and the upper crown 138. In some embodiments, the plurality of commissures 146 may be disposed distal of the plurality of stabilization arches 140 and proximal of the upper crown 138. In at least some embodiments, between circumferentially adjacent commissures of the plurality of commissures 146, the replacement heart valve implant 130 may be devoid of the expandable framework 132 at a longitudinal position radially outward of the free edges of the plurality of valve leaflets 134. As such, the free edges of the plurality of valve leaflets 134 may be free from direct contact with the expandable framework 132 as the plurality of valve leaflets 134 opens and/or closes.

In some embodiments, the connection portions of the plurality of valve leaflets 134 may also be referred to as commissural mounting tabs. In some embodiments, the connection portions may be disposed at least partially within a connection aperture defined and/or extending through the expandable framework 132 thereby coupling or attaching the plurality of valve leaflets 134 to the expandable framework 132. In some embodiments, the connection portions may be projections from their respective leaflet. In some embodiments, the connection portions may be integrally formed with its respective leaflet, such that the leaflet and connection portions are a single unitary and/or monolithic part or structure. In some embodiments, the connection portions of the leaflet can extend completely through the connection apertures, such as when the connection apertures extend completely through the expandable framework 132.

In some embodiments, the connection portions may encircle a portion of the expandable framework 132, such as when the connection portion contacts a strut at a location where the strut and/or the expandable framework 132 does not define a connection aperture. In some embodiments, the plurality of valve leaflets 134 and/or the connection portions may be attached to the expandable framework 132 using sutures, adhesives, or other suitable methods.

In some embodiments, the replacement heart valve implant 130 may include the inner skirt 142. In some embodiments, the inner skirt 142 may define a substantially tubular shape. The inner skirt 142 may be disposed on and/or extend along an inner surface (e.g., the luminal surface) of the expandable framework 132. In at least some embodiments, the inner skirt 142 may be fixedly attached to the expandable framework 132. The inner skirt 142 may direct fluid, such as blood, flowing through the replacement heart valve implant 130 toward the plurality of valve leaflets 134. In at least some embodiments, the inner skirt 142 may be fixedly attached to and/or integrally formed with the plurality of valve leaflets 134. The inner skirt 142 may ensure the fluid flows through the central lumen of the replacement heart valve implant 130 and does not flow around the plurality of valve leaflets 134 when they are in the closed position.

The inner skirt 142 may include a connection projection that extends from the inner skirt 142 and into one or more connection aperture. In some embodiments, the connection projection may extend around a portion of a strut and/or the expandable framework 132. In some embodiments, the connection projection may extend around a portion of a strut and into one or more connection aperture. In some embodiments, the connection projections may interact with the expandable framework 132 to attach or couple the inner skirt 142 to the expandable framework 132 through surface area contact and/or a form fitting configuration. In some embodiments, the connection projections may be attached to the expandable framework 132 using sutures, adhesives, or other suitable methods.

In some embodiments, the replacement heart valve implant 130 can include an outer skirt. In some embodiments, the outer skirt may define a substantially tubular shape. In some embodiments, the outer skirt may be disposed on the abluminal surface of the expandable framework 132. In some embodiments, the outer skirt may be disposed at and/or adjacent the lower crown 136. In some embodiments, the outer skirt may be disposed between the expandable framework 132 and the vessel wall in order to prevent fluid, such as blood, flowing around the replacement heart valve implant 130 and/or the expandable framework 132 in a downstream direction. The outer skirt may ensure the fluid flows through the replacement heart valve implant 130 and does not flow around the replacement heart valve implant 130, such as to ensure that the plurality of valve leaflets 134 can stop the flow of fluid when in the closed position.

The outer skirt may include a connection projection that extends from the outer skirt and into one or more connection aperture. In some embodiments, the connection projection may extend around a portion of a strut and/or the expandable framework 132. In some embodiments, the connection projection may extend around a portion of a strut and/or the expandable framework 132 and into one or more connection aperture. In some embodiments, the connection projections may interact with the expandable framework 132 to attach or couple the outer skirt to the expandable framework 132, such as through surface area contact or a form fitting configuration. In some embodiments, the connection projections may be attached to the expandable framework 132 using sutures, adhesives, or other suitable methods.

In some embodiments, the plurality of valve leaflets 134 may be comprised of a polymer, such as a thermoplastic polymer. In some embodiments, the plurality of valve leaflets 134 may include at least 50 percent by weight of a polymer. In some embodiments, the plurality of valve leaflets 134 may be formed from bovine pericardial or other living tissue. Other configurations and/or materials are also contemplated.

In some embodiments, the inner skirt 142 may include a polymer, such as a thermoplastic polymer. In some embodiments, the inner skirt 142 may include at least 50 percent by weight of a polymer. In some embodiments, the outer skirt may include a polymer, such as a thermoplastic polymer. In some embodiments, the outer skirt may include at least 50 percent by weight of a polymer. In some embodiments one or more of the plurality of valve leaflets 134, the inner skirt 142, and/or the outer skirt may be formed of the same polymer or polymers. In some embodiments, the polymer may be a polyurethane. In some embodiments, the inner skirt 142 and/or the outer skirt may be substantially impervious to fluid. In some embodiments, the inner skirt 142 and/or the outer skirt may be formed from a thin tissue (e.g., bovine pericardial, etc.). In some embodiments, the inner skirt 142 and/or the outer skirt may be formed from a coated fabric material. In some embodiments, the inner skirt 142 and/or the outer skirt may be formed from a nonporous and/or impermeable fabric material. Other configurations are also contemplated. Some suitable but non-limiting examples of materials that may be used to form the inner skirt 142 and/or the outer skirt including but not limited to polymers, composites, and the like, are described below.

In some embodiments, the inner skirt 142 may be coupled to the lower crown 136 and/or the upper crown 138. In some embodiments, the inner skirt 142 may be coupled only to the upper crown 138. In some embodiments, the outer skirt may be coupled to the lower crown 136 and/or the upper crown 138. In some embodiments, the outer skirt may be coupled only to the lower crown 136. In some embodiments, the plurality of valve leaflets 134 may be coupled to the expandable framework 132 at a position that is at or just below the plurality of stabilization arches 140 and above the upper crown 138.

In some embodiments, the expandable framework 132 and/or the replacement heart valve implant 130 may have an outer extent of about 23 millimeters (mm), about 25 mm, about 27 mm, about 30 mm, etc. in an unconstrained configuration (e.g., in the expanded configuration). In some embodiments, the expandable framework 132 and/or the replacement heart valve implant 130 may have an outer extent of about 10 mm, about 9 mm about 8 mm, about 7 mm, about 6 mm, etc. in the collapsed configuration. Other configurations are also contemplated.

In some embodiments, the inner skirt 142 and/or the outer skirt may seal one of, some of, a plurality of, or each of the plurality of interstices 133 formed in the expandable framework 132. In at least some embodiments, sealing the interstices may be considered to prevent fluid from flowing through the interstices from the luminal side of the expandable framework 132 to the abluminal side of the expandable framework 132. In some embodiments, the inner skirt 142 and/or the outer skirt may be attached to the expandable framework 132 and/or the plurality of frame struts 131 using one or more methods including but not limited to tying with sutures or filaments, adhesive bonding, melt bonding, embedding or over molding, welding, etc.

In use, a medical device system may generally be described as a catheter system that includes an implant delivery device for delivering a replacement heart valve implant 100 which may be coupled to the implant delivery device and disposed within a lumen of the implant delivery device during delivery of the replacement heart valve implant 100. The implant delivery device may include a proximal handle and an elongate shaft extending distally from the proximal handle. In some embodiments, the implant delivery device and/or the elongate shaft may include a proximal sheath and a distal sheath. The implant delivery device may include an inner shaft slidably disposed within a lumen of the elongate shaft. The inner shaft may be fixedly attached to the distal sheath. In some embodiments, the inner shaft may include a guidewire lumen extending therethrough. In some embodiments, the proximal handle may be configured to manipulate and/or translate the proximal sheath and/or the distal sheath relative to each other. In some embodiments, the proximal handle may be configured to manipulate and/or translate the inner shaft relative to the elongate shaft and/or the proximal sheath.

During delivery of the replacement heart valve implant 130, the replacement heart valve implant 130 may be disposed within the proximal sheath and/or the distal sheath in a collapsed configuration. In some embodiments, the proximal sheath and/or the distal sheath may collectively define a stent holding portion of the implant delivery device. In some embodiments, the stent holding portion may be configured to constrain the replacement heart valve implant 130 in the collapsed configuration. In some embodiments, the replacement heart valve implant 130 may be releasably coupled to the inner shaft.

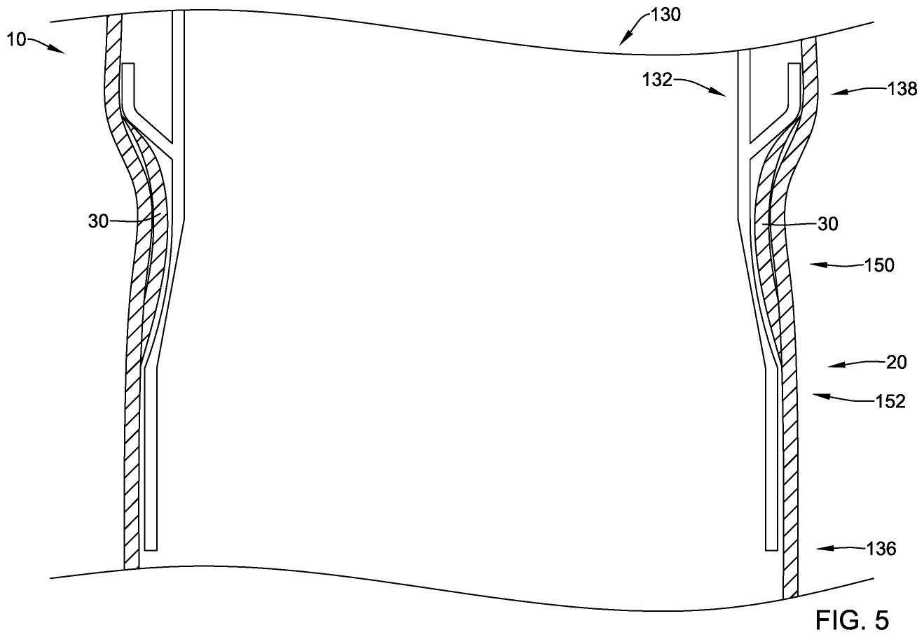

In use, the medical device system may be advanced percutaneously through the vasculature to a position adjacent to a treatment site. For example, the medical device system may be advanced through the vasculature and across the aortic arch to a position adjacent to a defective native heart valve 10. Alternative approaches to treat a defective aortic valve and/or other heart valve(s) are also contemplated with the medical device system. After navigating the implant delivery device and/or the stent holding portion to the treatment site, the proximal sheath and/or the distal sheath may be translated relative to each other to open the stent holding portion. When unconstrained by the stent holding portion, the replacement heart valve implant 130 may be configured to shift from the collapsed configuration to an expanded configuration. In at least some interventions, the replacement heart valve implant 130 may be deployed within the native heart valve 10 (e.g., the native heart valve 10 is left in place and not excised), as shown in FIG. 5 for example. It shall be noted that all features and/or elements of the replacement heart valve implant 130 are not illustrated in FIG. 5. Alternatively, the native heart valve 10 may be removed (such as through valvuloplasty, for example) and the replacement heart valve implant 130 may be deployed in its place as a replacement. Some suitable but non-limiting materials for the medical device system, implant delivery device, the proximal handle, the elongate shaft, the proximal sheath, the distal sheath, the inner shaft, the stent holding portion, and/or components or elements thereof, for example metallic materials and/or polymeric materials, are described below.

In FIG. 5, which illustrates the expandable framework 132 of the replacement heart valve implant 130 disposed within the annulus 20 of the native heart valve 10 (e.g., the aortic valve, etc.), the upper crown 138 is disposed downstream of the plurality of leaflets 30 of the native heart valve 10 and the lower crown 136 is disposed upstream of the plurality of leaflets 30 of the native heart valve 10. In at least some embodiments, the first circumferential row of x-connectors 150 may be disposed proximate the plurality of leaflets 30 of the native heart valve 10 and the second circumferential row of x-connectors 152 may be disposed against and/or adjacent to the one or more walls defining the annulus 20 of the native heart valve 10. In some embodiments, the second circumferential row of x-connectors 152 may be disposed upstream of the plurality of leaflets 30 of the native heart valve 10.

Bench testing has shown that the configuration(s) disclosed herein, wherein the thickness of at least some frame struts 131A of the plurality of frame struts 131 connecting the first circumferential row of x-connectors 150 to the second circumferential row of x-connectors 152 may be less than the thickness of other frame struts of the plurality of frame struts 131, may increase a radial outward force the expandable framework 132 exerts upon the annulus 20. In some embodiments, the thickness of the at least some frame struts 131A of the plurality of frame struts 131 connecting the first circumferential row of x-connectors 150 to the second circumferential row of x-connectors 152 may be reduced by about 25% compared to other frame struts of the plurality of frame struts 131. In some embodiments, the thickness of the at least some frame struts 131A of the plurality of frame struts 131 connecting the first circumferential row of x-connectors 150 to the second circumferential row of x-connectors 152 may be reduced by about 35% compared to other frame struts of the plurality of frame struts 131. In some embodiments, the thickness of the at least some frame struts 131A of the plurality of frame struts 131 connecting the first circumferential row of x-connectors 150 to the second circumferential row of x-connectors 152 may be reduced by about 50% compared to other frame struts of the plurality of frame struts 131.

Compared to an expandable framework lacking the reduced thickness of the at least some frame struts 131A of the plurality of frame struts 131, the expandable framework 132 may exert up to about 8-10% more radial outward force against the annulus 20. In some embodiments, compared to an expandable framework lacking the reduced thickness of the at least some frame struts 131A of the plurality of frame struts 131, the expandable framework 132 may exert up to about 6.5% more radial outward force against the annulus 20. In some embodiments, compared to an expandable framework lacking the reduced thickness of the at least some frame struts 131A of the plurality of frame struts 131, the expandable framework 132 may exert up to about 5% more radial outward force against the annulus 20. In some embodiments, compared to an expandable framework lacking the reduced thickness of the at least some frame struts 131A of the plurality of frame struts 131, the expandable framework 132 may exert up to about 3.5% more radial outward force against the annulus 20. For comparison, thinning all struts of the plurality of frame struts 131 (e.g., the entire expandable framework) by about 50% resulted in a 53-55% decrease in radial outward force exerted upon the annulus 20 by the expandable framework.

Accordingly, the configuration(s) disclosed herein unexpectedly produced an increase in radial outward force, while making the portion of the expandable framework 132 having a reduced thickness more compliant to the annulus 20. This configuration resulted in additional benefits over an unmodified expandable framework. For example, bench testing shows an increase in resistance to axial migration in a ventricular (upstream) direction of about 12.3%. Additionally, bench testing shows an increase in the amount of force required to axially translate the expandable framework 132 in an aortic (downstream) direction from an average of about 4.5 pounds of force to about 10.1 pounds of force in one test and from an average of about 3.5 pounds of force to about 9.9 pounds of force in a second test. The first and second tests differed in the diameter of the fixture, which the second test having a slightly larger fixture diameter (about 4%) than the first test. As such, the disclosed configuration(s) have made undesired axial translation of the expandable framework 132 within the annulus 20 more difficult.

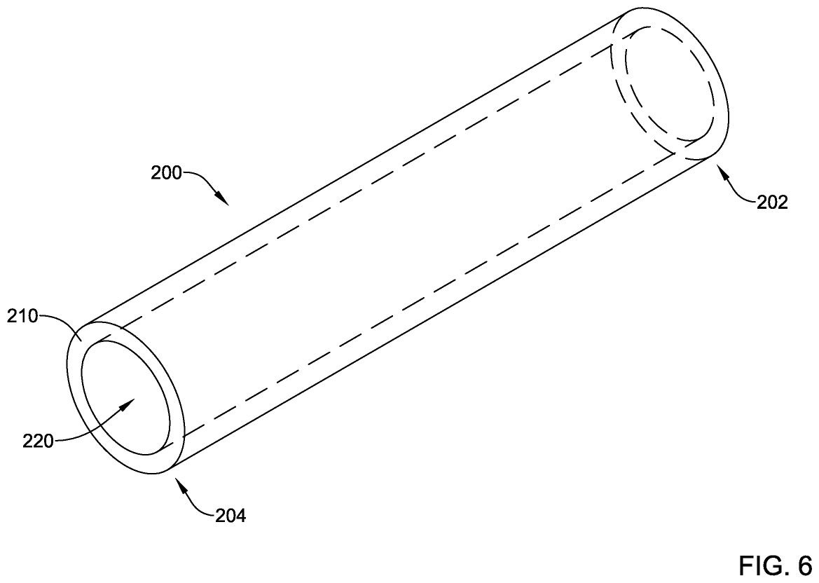

FIGS. 6-8 illustrates aspects of a method of manufacturing the expandable framework 132 of the replacement heart valve implant 130. As discussed herein, the expandable framework 132 may be formed from a tubular member 200, shown in FIG. 6. In some embodiments, the tubular member 200 may be a metallic tubular member. Other configurations are also contemplated. The tubular member 200 may include a wall 210 defining a lumen 220 extending from a proximal end 202 of the tubular member 200 to a distal end 204 of the tubular member 200.

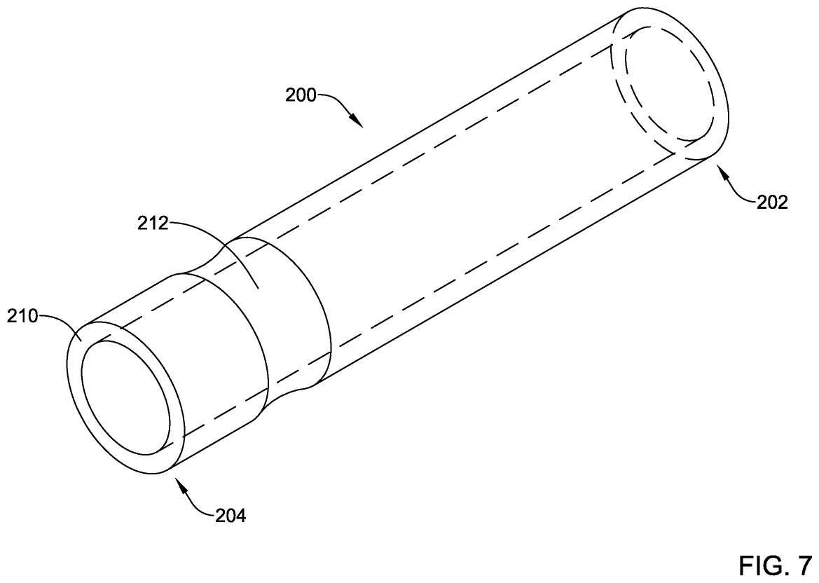

The method may include removing a portion of the wall 210 of the tubular member 200 to form an area of reduced thickness 212, as seen in FIG. 7. In some embodiments, the portion of the wall 210 of the tubular member 200 forming the area of reduced thickness 212 may be varied in thickness, may be stepped, may be tapered, etc. In some embodiments, the area of reduced thickness 212 may extend radially inward from an outer surface of the wall 210 of the tubular member 200. In some embodiments, the area of reduced thickness 212 may be formed by one or more appropriate methods including but not limited to machining, grinding, chemical dissolution, etc. In at least some embodiments, the area of reduced thickness 212 may be disposed closer to the distal end 204 than the proximal end 202.

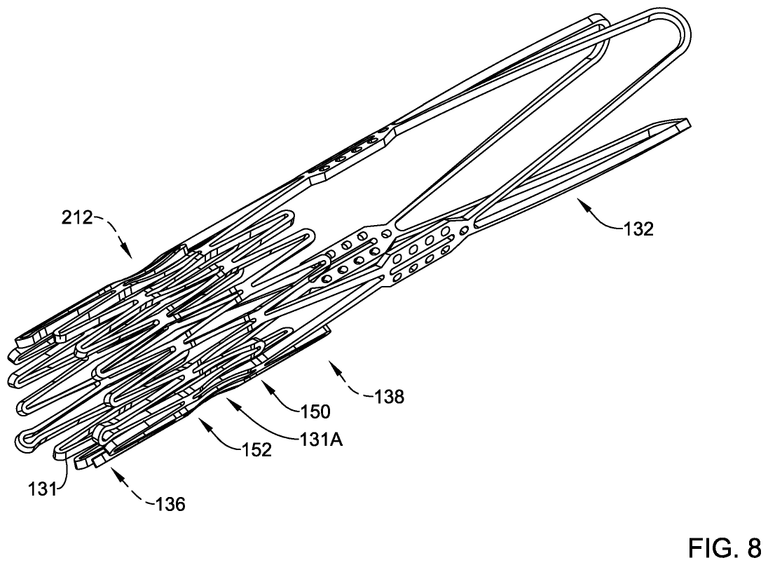

As may be seen in from FIG. 8, which shows the expandable framework 132 in an as-cut configuration, the area of reduced thickness 212 shown in FIG. 7 may correspond to the at least some frame struts 131A of the plurality of frame struts 131 connect the first circumferential row of x-connectors 150 to the second circumferential row of x-connectors 152. Subsequently, the expandable framework 132 may be formed and/or heat set to define the upper crown 138 and/or the lower crown 136 in the expanded configuration (e.g., FIG. 3).

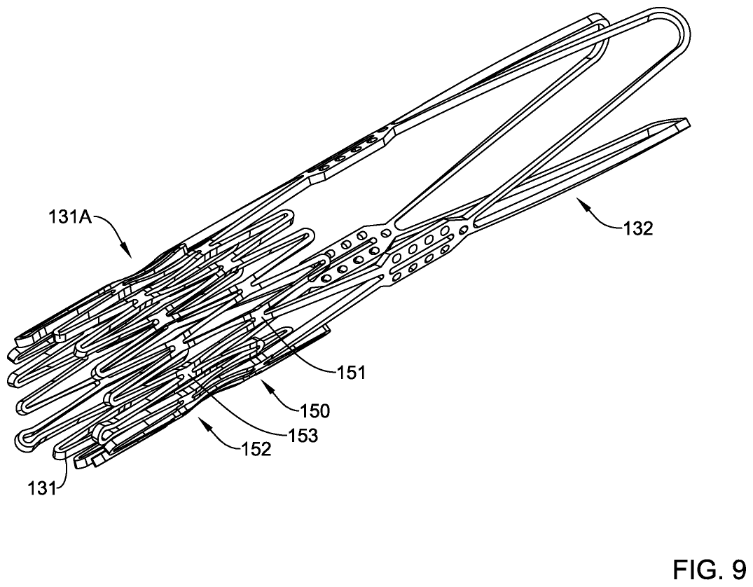

FIG. 9 illustrates an alternative configuration of the expandable framework 132 in the as-cut configuration. Similar to FIG. 8 above, the area of reduced thickness 212 shown in FIG. 7 may correspond to the at least some frame struts 131A of the plurality of frame struts 131 connect the first circumferential row of x-connectors 150 to the second circumferential row of x-connectors 152. Additionally, x-connectors 151 of the first circumferential row of x-connectors 150 and/or x-connectors 153 of the second circumferential row of x-connectors 152 may have additional material removed from the thickness of the first circumferential row of x-connectors 150 and/or the second circumferential row of x-connectors 152, respectively. As such, a shallow notch may be formed over and/or including the x-connectors 151 and/or the x-connectors 153. In some embodiments, the shallow notch may be formed by one or more suitable methods including but not limited to machining, grinding, etc. Other configurations are also contemplated. For example, the x-connectors 151 and/or the x-connectors 153 may be tapered and/or stepped in thickness compared to other frame struts of the plurality of frame struts 131.

The materials that can be used for the various components of the medical device system and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the expandable framework, the inner skirt, the outer skirt, the plurality of leaflets, and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof, may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An expandable framework for use in a replacement heart valve implant, comprising:
a plurality of frame struts defining a lattice structure around a central longitudinal axis, each frame strut having a thickness in a radial direction from the central longitudinal axis;
wherein the plurality of frame struts defines a lower crown proximate an inflow end of the lattice structure and upper crown proximate an outflow end of the lattice structure and a plurality of stabilization arches extending downstream from the outflow end of the lattice structure;
wherein the lattice structure includes a first circumferential row of x-connectors upstream of the upper crown and a second circumferential row of x-connectors downstream of the lower crown;
wherein the thickness of at least some frame struts connecting the first circumferential row of x-connectors to the second circumferential row of x-connectors is less than the thickness of other frame struts of the plurality of frame struts.

2. The expandable framework of claim 1, wherein the thickness of the at least some frame struts connecting the first circumferential row of x-connectors to the second circumferential row of x-connectors is less than the thickness of frame struts downstream of the first circumferential row of x-connectors.

3. The expandable framework of claim 2, wherein the thickness of the at least some frame struts connecting the first circumferential row of x-connectors to the second circumferential row of x-connectors is less than the thickness of all frame struts downstream of the first circumferential row of x-connectors.

4. The expandable framework of claim 1, wherein the thickness of the at least some frame struts connecting the first circumferential row of x-connectors to the second circumferential row of x-connectors is less than the thickness of frame struts upstream of the second circumferential row of x-connectors.

5. The expandable framework of claim 4, wherein the thickness of the at least some frame struts connecting the first circumferential row of x-connectors to the second circumferential row of x-connectors is less than the thickness of all frame struts upstream of the second circumferential row of x-connectors.

6. The expandable framework of claim 1, wherein the thickness of the at least some frame struts connecting the first circumferential row of x-connectors to the second circumferential row of x-connectors tapers radially inward toward a medial portion of the at least some frame struts connecting the first circumferential row of x-connectors to the second circumferential row of x-connectors.

7. The expandable framework of claim 1, wherein the thickness of the at least some frame struts connecting the first circumferential row of x-connectors to the second circumferential row of x-connectors tapers from a first thickness at a first position adjacent the first circumferential row of x-connectors and a second thickness at a second position adjacent the second circumferential row of x-connectors to a minimum thickness at a third position disposed longitudinally between the first position and the second position;
wherein the minimum thickness is less than the first thickness and the second thickness.

8. The expandable framework of claim 1, wherein the at least some frame struts connecting the first circumferential row of x-connectors to the second circumferential row of x-connectors includes all frame struts of the plurality of frame struts directly connecting the first circumferential row of x-connectors to the second circumferential row of x-connectors.

9. The expandable framework of claim 1, wherein the upper crown defines a first maximum outer extent of the lattice structure, and the lower crown defines a second maximum outer extent of the lattice structure;
wherein the at least some frame struts connecting the first circumferential row of x-connectors to the second circumferential row of x-connectors defines a third maximum outer extent of the lattice structure less than the first maximum outer extent and the second maximum outer extent.

10. A replacement heart valve implant, comprising:
an expandable framework comprising a plurality of frame struts defining a lattice structure around a central longitudinal axis, each frame strut having a thickness in a radial direction from the central longitudinal axis; and
a plurality of valve leaflets coupled to the expandable framework;
wherein the plurality of frame struts defines a lower crown proximate an inflow end of the lattice structure and upper crown proximate an outflow end of the lattice structure and a plurality of stabilization arches extending downstream from the outflow end of the lattice structure;
wherein the lattice structure includes a first circumferential row of x-connectors upstream of the upper crown and a second circumferential row of x-connectors downstream of the lower crown;
wherein the thickness of at least some frame struts connecting the first circumferential row of x-connectors to the second circumferential row of x-connectors is less than the thickness of other frame struts of the plurality of frame struts.

11. The replacement heart valve implant of claim 10, wherein the plurality of valve leaflets is configured to substantially restrict fluid from flowing through the replacement heart valve implant in a closed position.

12. The replacement heart valve implant of claim 10, wherein the plurality of valve leaflets is fixedly attached to the expandable framework at a plurality of commissures disposed adjacent the plurality of stabilization arches.

13. The replacement heart valve implant of claim 12, wherein the plurality of commissures is disposed longitudinally between the plurality of stabilization arches and the upper crown.

14. The replacement heart valve implant of claim 10, further comprising an outer skirt disposed on an abluminal surface of the expandable framework.

15. An expandable framework for use in a replacement heart valve implant, comprising:
a plurality of frame struts defining a lattice structure around a central longitudinal axis, each frame strut having a thickness in a radial direction from the central longitudinal axis;
wherein the plurality of frame struts defines a lower crown proximate an inflow end of the lattice structure and upper crown proximate an outflow end of the lattice structure and a plurality of stabilization arches extending downstream from the outflow end of the lattice structure;
wherein the lattice structure includes a first circumferential row of x-connectors and a second circumferential row of x-connectors longitudinally spaced apart from the first circumferential row of x-connectors;
wherein the thickness of at least some frame struts directly connecting the first circumferential row of x-connectors to the second circumferential row of x-connectors varies.

16. The expandable framework of claim 15, wherein the thickness of at least some frame struts directly connecting the first circumferential row of x-connectors to the second circumferential row of x-connectors varies in a longitudinal direction.

17. The expandable framework of claim 15, wherein the thickness of each frame strut directly connecting the first circumferential row of x-connectors to the second circumferential row of x-connectors varies.

18. The expandable framework of claim 15, wherein the thickness of each frame strut directly connecting the first circumferential row of x-connectors to the second circumferential row of x-connectors varies in a longitudinal direction.

19. The expandable framework of claim 15, wherein the thickness of the at least some frame struts directly connecting the first circumferential row of x-connectors to the second circumferential row of x-connectors tapers radially inward toward a medial portion of the at least some frame struts directly connecting the first circumferential row of x-connectors to the second circumferential row of x-connectors.

20. The expandable framework of claim 15, wherein the thickness of the at least some frame struts directly connecting the first circumferential row of x-connectors to the second circumferential row of x-connectors tapers radially inward from the first circumferential row of x-connectors toward the second circumferential row of x-connectors and the thickness of the at least some frame struts directly connecting the first circumferential row of x-connectors to the second circumferential row of x-connectors tapers radially inward from the second circumferential row of x-connectors toward the first circumferential row of x-connectors.
